(19) United States
(12) Patent Application Publication
(10) Pub. No.: US 2025/0272842 A1
(43) Pub. Date: Aug. 28, 2025

(54) Systems And Methods For Processing Images Related To Boundaries

(71) Applicant: CLIMATE LLC, Saint Louis, MO (US)

(72) Inventors: Angeles CASAS, San Francisco, CA (US); Yu LIU, Newark, CA (US); Pratik SHRIVASTAVA, Saint Louis, MO (US); Jun XIONG, Orinda, CA (US)

(21) Appl. No.: 19/064,529

(22) Filed: Feb. 26, 2025

Related U.S. Application Data
(60) Provisional application No. 63/558,564, filed on Feb. 27, 2024.

(51) Int. Cl. G06T 7/10; G06V 20/10

(52) U.S. Cl. CPC G06T 7/10; G06V 20/188; G06T 2207/10024

(57) ABSTRACT

Systems and methods are provided for processing images related to boundaries. An example computer-implemented method includes accessing an image data set, which includes multiple images of an agricultural field; masking the image data set based on one or more criteria; calculating, by a computing device, one or more composites from the image data set; generating, by the computing device, using a model, a segmentation of each of the one or more composites, based on the one or more composites; and combining the segmentation(s) for the one or more composites into a field boundary for the agricultural field.

### CROSS-REFERENCE TO RELATED APPLICATION

This application claims the benefit of, and priority to, U.S. Provisional Application No. 63/558,564, filed Feb. 27, 2024. The entire disclosure of the above application is incorporated herein by reference.

### FIELD

The present disclosure generally relates to systems and methods for processing images related to boundaries.

### BACKGROUND

This section provides background information related to the present disclosure which is not necessarily prior art.

Images of agricultural fields are known to be captured in various manners, including, for example, by satellites, unmanned and manned aerial vehicles, etc. In addition, the images are known to have specific characteristics based on the manner in which the images are captured. Further, the images of the agricultural fields, once captured, are known to be processed, and then used to determine features of the agricultural fields, such as, for example, growth stages of crops planted in the agricultural fields.

### SUMMARY

This section provides a general summary of the disclosure and is not a comprehensive disclosure of its full scope or all of its features.

Example embodiments of the present disclosure generally relate to computer-implemented methods for use in processing images related to boundaries. In one example embodiment, such a method generally includes: accessing an image data set, which includes multiple images of an agricultural field; masking the image data set based on one or more criteria; calculating, by a computing device, one or more composites from the image data set; generating, by the computing device, using a model, a segmentation of each of the one or more composites, based on the one or more composites; and combining the segmentation(s) for the one or more composites into a field boundary for the agricultural field.

Example embodiments of the present disclosure also generally relate to systems for use in processing images related to boundaries. In one example embodiment, such a system comprises at least one computing device configured to: access an image data set, which includes multiple images of an agricultural field; mask the image data set based on one or more criteria; calculate one or more composites from the image data set; generate, using a model, a segmentation of each of the one or more composites, based on the one or more composites; and combine the segmentation(s) for the one or more composites into a field boundary for the agricultural field.

Example embodiments of the present disclosure also generally relate to non-transitory computer readable storage media including computer-executable instructions for processing images related to boundaries, which when executed by at least one processor, cause the at least one processor to perform one or more of the above operations.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 illustrates an example system of the present disclosure for use in identifying field boundaries based on one or more image data sets;

FIG. 2 illustrates example segmentation of multiple different composites of an example image data set;

FIG. 3 illustrates an example method that may be used and/or implemented in the system of FIG. 1, for identifying field boundaries based on one or more composites calculated from one or more image data sets;

FIGS. 4A and 4B illustrate an example logical organization of sets of instructions in main memory of a computing device (or computer system) when an example application is loaded on the computing device for execution, which may be used in connection with the system of FIG. 1 and/or the method of FIG. 3; and

FIG. 5 is a block diagram that illustrates an example computing device (or computer system) upon which embodiments of the system of FIG. 1 and/or the method of FIG. 3 may be implemented.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

Images may be used to identify, and obtain insights into, different crop characteristics within agricultural fields in which the crops are planted/growing. However, a lack of accurate (and/or consistent) boundaries associated with the agricultural fields limits the accuracy of the underlying analysis. That is, when a field boundary is inaccurate (or inconsistently determined), the underlying data which is supposed to be representative of that field is either overly inclusive, or underinclusive as to the data analyzed. In this manner, crop metrics may be incorrect. And, decisions based on the incorrect metrics may be likewise incorrect. As such, a technical problem exists in accurately identifying field boundaries across different fields with different sizes, different crops and/or in different regions.

Uniquely, the systems and methods herein enable identifying agricultural field boundaries based on processing images of the agricultural fields (e.g., images from satellites, aerial vehicles, etc.).

In particular, certain images of an area of interest (e.g., an agricultural field, etc.) are compiled into an image data set. An agricultural computer system optionally masks certain of the images and then generates composites of the remaining images in the image data set. One or more of the composites is input to a model along with one or more prompts, whereby a segmentation of the area of interest is determined. The segmentation, or multiple segmentations, are compiled into a field boundary for the area of interest. The field boundary may then be used in various operations related to the agricultural field, including, without limitation, processing of data related to the agricultural field; determining performance of the agricultural field; identifying planting, treatment, and harvesting operations for the agricultural field; etc.

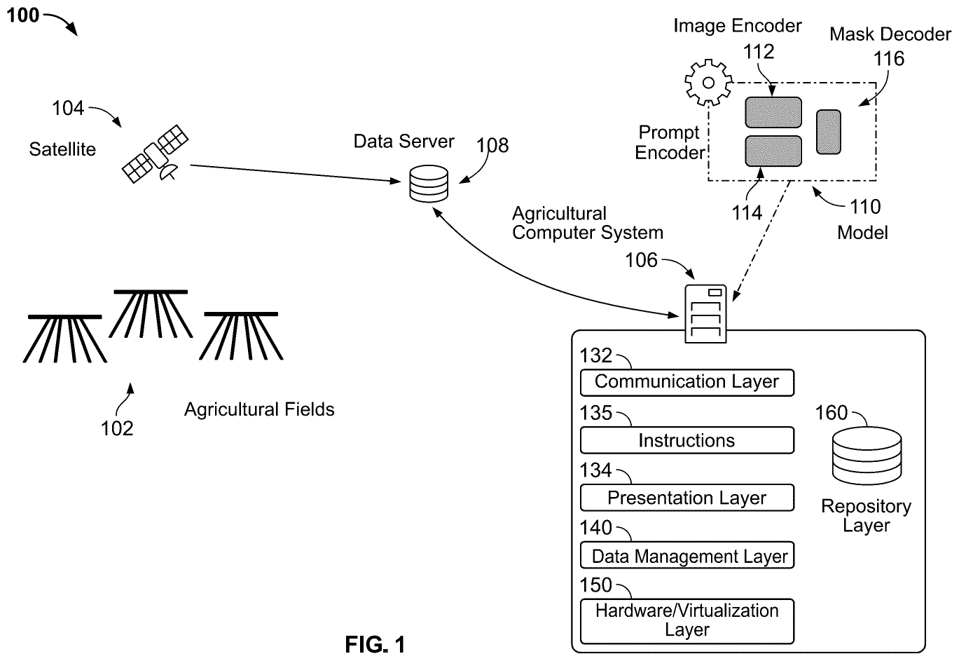

FIG. 1 illustrates an example system 100 in which one or more aspect(s) of the present disclosure may be implemented. Although the system 100 is presented in one arrangement, other embodiments may include the parts of the system 100 (or other parts) arranged otherwise depending on, for example, types of crops, types and/or locations of agricultural fields, and/or privacy and/or data requirements, etc.

The system 100 generally includes a plurality of agricultural fields 102, which may be of any size, acreage, etc. The agricultural fields 102 should be understood to include plots, sub-fields, etc. (broadly, growing spaces), wherein each of the agricultural fields 102 is a physical location, in which crops are planted, grown, and/or harvested, etc. The agricultural fields 102 are defined by boundaries, or field boundaries. The field boundaries delineate the agricultural fields 102 from one another and their surroundings. The agricultural fields 102 may be associated with one or more growers, which, in turn, are responsible for decisions related to the agricultural field(s) 102. The grower(s) may include the owner(s), operator(s) or other user(s) associated with the agricultural fields 102.

Additionally, in this example embodiment, the agricultural fields 102 include one or more crops planted/growing therein (e.g., single crops, multiple different crops, etc.), for example, wheat, corn—white, corn—popcorn, soybeans, soycorn, cereal, etc., or another suitable crop(s), or combination of crops. The crop(s) are planted in the agricultural fields 102 at a planting time and harvested from the agricultural fields 102 at a harvest time. The planting and harvesting of the crop(s) defines a growing season (or seasons), which may be various times of the year depending on the crop(s) and location(s) of the agricultural fields 102, etc.

As shown in FIG. 1, in the example embodiment, the system 100 further includes a satellite 104, an agricultural computer system 106, and a data server 108 coupled in communication with the satellite 104 and the agricultural computer system 106.

In general, in the example embodiment, the agricultural computer system 106 is configured to identify field boundaries of the agricultural fields 102 based on image data captured by the satellite 104 and stored in the data server 108.

Specifically, as shown in FIG. 1, the satellite 104 is disposed in orbit about the Earth, which includes the agricultural fields 102. The satellite 104 is configured to capture images or image data representative of one or more of the agricultural fields 102, and other agricultural fields. As indicated above, the satellite 104 may be part of a collection of satellites (including multiple companion satellites), which collectively orbit the Earth and capture images thereof. Example collections of satellites, which may be represented herein by the satellite 104, include the Landsat satellite system, the Sentinel-2 satellite system, National Agriculture Imagery, PlanetScope Imagery, etc.

In this example embodiment, satellite 102 is configured to capture one or more images, at an interval of once per N days, for example, where N may include one day (i.e., daily), two days, five days, seven days (i.e., weekly), ten days, 15 days, 20 days, 25 days, 30 days, numbers of days therebetween, more than 30 days, etc., thereby defining a temporal resolution of satellite images of (or captured for) the agricultural fields 102. More generally, the images may be captured at one or more regular or irregular intervals. Also, the satellite images may include different resolutions, depending on the particular type of satellite 104 employed in capturing the images. In one example, the images may include a resolution of about 10 meters by 10 meters per pixel, while in another example, the images may include a solution of about three meters by three meters, etc. In other examples, the resolution may be higher, or lower, again, depending on the satellite 104 (or other apparatus) employed in capturing the images.

As the image(s) are captured, or at one or more intervals, the satellite 104 is configured to transmit the images to the data sever 108, either directly, or via the agricultural computer system 106.

The data server 108 is configured to, in turn, receive the image data from the satellite 104 and to store the image data in memory thereof, in a suitable manner. In this way, the data sever 108 includes multiple images of the agricultural fields 102 over a time period (e.g., as a time series of images, etc.), which may correspond to the year, multiple years, growing season(s), etc., depending on the start date and duration of the given time period. In general, the data server 108 includes images once per N days, throughout the year, including spring, summer, fall, and winter seasons. The images may be organized, in the data server 108, by date (e.g., year, etc.) and/or by the physical location represented by the images (e.g., North America, Iowa, Nebraska, Pacific Northwest, Latin America, Europe, etc.). In one example, the data server 108 may be part of, or may be associated with, or may be in communication with the National Agriculture Imagery Program (NAIP), which acquires aerial imagery during the agricultural growing seasons in the United States and other regions, as appropriate.

In addition to the image data, the data server 108 may store a number of ground truths, including, without limitation, the verified field boundaries for one or more different agricultural fields, i.e., other than agricultural fields 102. The verified field boundaries include specific geometries, specific to the different agricultural fields, to delineate the agricultural fields from other agricultural fields and/or the surroundings thereof. It should be appreciated that the image data includes images of the different agricultural fields over one or more intervals, whereby the specific geometries are associated with and/or linked to the image data. The combination of the image data and the field boundary ground truths define a training data set to be used as described below.

In this example embodiment, the agricultural computer system 106 includes a segment anything model (SAM) 110. In connection therewith, with regard to object segmentation, the model 110 is configured to generate object masks from various input prompts, including points or shapes (e.g., box shapes, etc.), across a wide range of images. For instance, in using the model 110 for image segmentation, visual prompts may be provided to the model 110 in the form of point annotations or bounding boxes on a given input image, thereby instructing (or guiding) the model 110 to segment desired portions, regions, objects, etc. within the image.

In the illustrated embodiment, the model 110 includes an image encoder 112, a prompt encoder 114, and a mask decoder 116. The image encoder 112 is configured to generate one time imaging embeddings for input images to the model 110, and the prompt encoder 114 is configured to encode prompt(s) into an embedding vector. In some examples, each prompt includes a point within the agricultural field of an image. In other embodiments, each prompt includes a box (or other shape) within the agricultural field of an image. Further, the mask decoder 116 is configured to predict the segmentation masks indicative of the field boundaries of the agricultural field, based on the embeddings from both the image and prompt encoders 112, 114. The mask decoder 116 is configured to, then, map the image embedding, the prompt embeddings, and an output to a mask indicative of the field boundaries. This configuration of the model 110 facilitates prompt-based segmentation, whereby the model 110 is operable to produce accurate masks based on spatial and/or textual cues. An example segmentation model, as implemented by Meta Inc., is available at https://github.com/facebookresearch/segment-anything.

As noted, in some example embodiments, the segmentation model is prompted by one or more points (e.g., a pixel coordinate or coordinates, etc.). That is, the prompt (as the starting point for segmentation) may be a point within an area of interest (AOI) whose boundaries are to be generated by the segmentation model (e.g., indicating to the model 110 that the point and the region around the point in an image should be included in the resulting mask for the image, etc.). Additionally, in some example embodiments, the segmentation model may utilize a box-prompt strategy, based on any suitable data related to the AOI. In one example, the box-prompt strategy leverages crop-specific land cover datasets (e.g., the Cropland Data Layer (CDL) from the United States Department of Agriculture (USDA), etc.), whereby different crops, for example, are used to prompt the model. Other types of data may be used to prompt the model, especially where the data is consistent in segments of the AOI. In such embodiments, the segmentation model may be prompted by one or more shapes (e.g., a box, etc.) defined within an AOI of an image whose boundaries are to be generated by the segmentation model (e.g., the model 110 will use the bounding box (or boxes) as a prompt (or prompts) to segment the objects or regions within the given boundaries of the box(es), etc.).

Prior to identifying field boundaries, the model 110 is trained based on the training data set defined above. In particular, in this exemplary embodiment, the model 110 is trained, through use of the Segment Anything 1 Billion Mask (SA-1B) dataset, which includes natural scenes, urban environments, medical imagery, satellite images, etc. (e.g., including over 11 million images and over 1.1 billion object masks, etc.). Such use of the SA-1B dataset provides the model 110 with zero-shot performance capabilities, and allows the model 110 to effect multiple segmentation tasks without the need for task-specific training. That said, in this example embodiment, the training data used for the model 110 (e.g., the SA-1B dataset, etc.) is not specific to field boundaries, yet in other embodiments, the training data may be specific to field boundaries.

After training the model 110, in connection with identifying one or more field boundaries of the agricultural fields 102, the agricultural computer system 106 is configured to identify an area of interest (AOI) and a time of interests (TOI). The AOI includes, without limitation, the agricultural fields 102, and the TOI may include, without limitations, one or more seasons associated with the agricultural field 102. In this particular example, the TOI includes the spring, summer, and fall seasons for the particular locations represented in the images. That is, for example, in North America, the winter months, including at least January and February, are excluded from the TOI, while in South America the winter months, including at least July and August, are excluded from the TOI. In other words, the TOI may be a subset of the images captured by the satellite 104.

It should be understood that the AOI and/or TOI may be defined by a user of the agricultural computer system 106, which may include a person associated with the agricultural computer system 106 and/or the agricultural fields 102.

In this example embodiment, the agricultural computer system 106 is configured to access the image data from the data server 108 consistent with the TOI and the AOI, which is referred to herein as an image data set. The image data set includes multiple images as described above, where each image includes RGBN (i.e., red, green, blue, NIR) data. That is, the images include specific wavelength band data or band data, per pixel per image, which relate to the color RED (e.g., having wavelengths ranging between about 635 nm and about 700 nm, etc.), the color BLUE (e.g., having wavelengths ranging between about 490 nm and about 550 nm, etc.), the color GREEN (e.g., having wavelengths ranging between about 520 nm and about 560 nm, etc.), and near infrared (NIR) (e.g., having wavelengths ranging between about 800 nm and about 2500 nm, etc.), etc. It should be appreciated that more or less band data may be identified from (or included in) the images (and/or image data) in other embodiments.

In addition, in example embodiments, the agricultural computer system 106 is also configured to access data for the TOI and the AOI, which indicates one or more parameters of the same. One example of such data includes crop-specific land cover data consistent with the TOI and AOI. In one particular embodiment, the crop-specific land cover data includes CDL data accessed from the USDA. The CDL includes annually captured, geo-referenced, crop-specific land cover data produced using satellite imagery and agricultural ground reference data, as provided by the USDA. The crop-specific land cover data is indicative of, for example, the type(s) of crops included in the AOI. Other data may include other parameters associated with the AOI.

As indicated above, in this example embodiment, the agricultural computer system 106 is configured to identify the field boundaries of the agricultural field 102, which is represented by the accessed image data of the AOI (and additionally, in some examples, by the accessed CDL data).

Initially, the agricultural computer system 106 is configured to perform masking or filtering of the accessed images of the image data set. In particular, in this example, each pixel of each image is assigned a value, which includes, without limitation, 2 for dark/shadow, 4 for vegetation, 5 for not-vegetation, 6 for water, and 7 for unclassified. The values are based on SCL (Scene Classification) bands of the Sentinel-2 Level-2A (L2A), where other values are considered as “invalid pixels” and are consequently masked out. The agricultural computer system 106 is configured to count the number of valid pixels (and/or value for all of the pixels) in each image and to compare the count to a threshold (e.g., 75%, 85%, 95%, etc.). The agricultural computer system 106 is further configured to select only the images having valid pixel counts that satisfy the threshold (e.g., above 85%, etc.), while other images are masked or filtered from the image data set.

It should be appreciated that a number of images may be retained regardless of the mask based on the valid pixel count. For example, the agricultural computer system 106 may be configured to select the top N images, where N is an integer, when less than N images are selected based on the threshold defined above. For example, at least five, ten, fifteen, etc., images may be retained based on the pixel value count, either retained/eliminated based on the threshold or retained based on the minimum N images, etc.

Next, the agricultural computer system 106 is configured to perform a further masking or filtering of the image data set based on “blurriness” of images. In particular, agricultural computer system 106 is configured to calculate a blur effective metric for each of the images included in the image data set. The blur effect metric may include, for example, generating a blurred image based on a low pass filter, and then measuring the different in intensities between the origin and the blurred image. And, finally, the agricultural computer system 106 compares the intensity variations/differences in the two images (i.e., the original and the blurred image) to estimate the blur effect metric. The agricultural computer system 106 is configured to select only the images having a blur effect metric that satisfies a defined threshold (e.g., 0.3, etc.), while other images are masked or filtered from the image data set.

It should be appreciated, again, that a number of images may be retained regardless of the mask based on the blur effect metric. For example, the agricultural computer system 106 may be configured to select the top M images, where M is an integer, when less than M images are selected based on the threshold defined above. For example, at least five, ten, fifteen, etc., images may be retained based on the blur effect metric, either retained/eliminated based on the threshold or retained based on the minimum M images, etc.

With continued reference to FIG. 1, the agricultural computer system 106 is then configured to identify one or more prompts in the images (e.g., point prompts, box prompts, etc.).

In some example embodiments, the agricultural computer system 106 is configured to flag edges of the agricultural field 102 included in the images to avoid an errant prompt. That is, the agricultural computer system 106 is configured to calculate canny edges for the individual images (e.g., with sigma=3, etc.), to identify edge composites, to normalize intensity levels of edge composites, and to split intensity maps into binary, where 0 is background and 1 is the edge zones. In this way, the edges included within the images are defined. Next the agricultural computer system 106 is configured to identify a centroid of each of the images (or other boundary) and to determine if the centroid is within the edge zones. If not, the agricultural computer system 106 is then configured to identify the centroid as a prompt for the given image (e.g., a point prompt for the given mage, etc.). Conversely, if so, the agricultural computer system 106 is then configured to search nearby pixels in the horizontal and or vertical direction for background. For example, the agricultural computer system 106 walks out from the centroid in both horizontal and vertical directions until a background pixel is identified, and then, the agricultural computer system 106 determines which of the identified background pixels is closest to the centroid. The closest of the identified pixels is identified as a prompt for the image (e.g., a point prompt for the given mage, etc.).

In some example embodiments, as discussed above, the agricultural computer system 106 is configured to utilize a box-prompt strategy in connection with identifying the prompts in the images. For instance, the agricultural computer system 106 may instead apply a box-prompt strategy in which a bounding box may be defined around the centroid of the image or around the closest identified pixel, thereby defining a box (or other shape) prompt in the image (in lieu of identifying the centroid or pixel as a point prompt, etc.). Alternatively, in some example embodiments, in utilizing the box-prompt strategy, the agricultural computer system 106 may be configured to identify the CDL data (e.g., a map of such data, etc.) in the same resolution/project as the RGB clip of the given image and ensure that they are pixel-level aligned. The agricultural computer system 106 may also relabel the CDL data for the field (or image data set including the image of the field), for instance, to account for patches with the same crop type being different (e.g., if they are separate patches, etc.). Then, for each crop patch, the agricultural computer system 106 may create a box-prompt to represent the spatial extent of the crop patch, i.e., the type of crop (as the prompt for the image).

It should be appreciated that other techniques may be employed to identify prompt(s) within the background of the images, to avoid edges between ones of the agricultural fields, etc. Techniques may include, for example, manual selection of prompts (e.g., by a user viewing the images, etc.), or automatic selection of prompts. For automatic selection, the agricultural computer system 106 is configured to start at either the centroid of the area of interest (AOI) and moving from the centroid until a suitable prompt is found (e.g., where the suitable is located in the background of the images, etc.), or randomly generating one or more prompts near the centroid and then filtering out those that intersect with linear objects. It should be appreciated that other suitable techniques may be employed for identifying prompts.

The agricultural computer system 106 is then configured to calculate one or more different composites of the images included in the masked image data set.

In particular, in this example embodiment, the agricultural computer system 106 is configured to calculate the RGB mean, the RGB median and the RGB standard deviation for the image pixels, over the masked image data set (i.e., over the TOI). In doing so, the agricultural computer system 106 defines three composite images. Also, the agricultural computer system 106 is configured to compute one or more example indices, as defined in the equations below. For example, the indices may include vegetative indices, such as the Normalized Difference Vegetation Index (NDVI), the Bare Soil Index (BSI), the Enhanced Vegetation Index 2 (EVI2), and the Modified Soil Adjusted Vegetation Index (MSAVI), etc.

$$N V D I = \frac{( Nir - Red )}{( Nir + Red )}$$

$$B S I = \frac{( ( Red + Blue ) - Green )}{( ( Red + Blue ) + Green )}$$

$$E V I 2 = 2.5 * \frac{( Nir - Red )}{( Nir + 6 * Red - 7.5 * Blue + 1 )}$$

$$M S A V I = \frac{( 2 * Nir + 1 - \sqrt{{( 2 * Nir + 1 )}^{2} - 8 * ( Nir - Red )} )}{2}$$

It should be appreciated that the above-described indices are example only. Other indices or composites based on the band data, or combinations thereof, whether indicative of vegetation or not, may be generated by the agricultural computer system 106 in other system embodiments (and then appended to the data structure, etc.).

The agricultural computer system 106 is configured to define additional composite images based on the indices. For example, the agricultural computer system 106 is configured to define a composite based on NVDI in the 5^{th} percentile, and also a composite based on NDVI in the 95% percentile, and still further a composite based on a delta between the 5^{th} percentile and in the 95^{th} percentile. In doing so, the agricultural computer system 106 defines three additional composite images.

Consistent with the above, then, six composite images from the masked image data set are compiled by the agricultural computer system 106.

It should be appreciated that more or less composite images including different combinations of RGB, indices, or other composites, etc., may be employed in other system embodiments. For example, the agricultural computer system 106 may be configured to impose contrast adjustments to RGB and/or NVDI values for the images, prior to segmentation below. The adjustment(s) is (are) accomplished using the contrast limited adaptive histogram equalization (CLAHE), whereby the boundary information derived from the various composites of the images is effectively enhanced.

With continued reference to FIG. 1, the agricultural computer system 106 is configured to input the composite images and the identified prompt(s) to the model 110.

It should be understood that one or more images from the masked image data set may additionally or alternatively be input to the model 110 in other embodiments. For example, a late growing season image (i.e., within an interval of harvest) may be input to the model 110, along with the NDVI delta composite, to provide two segmentations of the AOI. It should be understood therefore that any combination of one or more composites, images, etc., may be input to the model 110 in other embodiments to generate one or ore segmentations of the AOI.

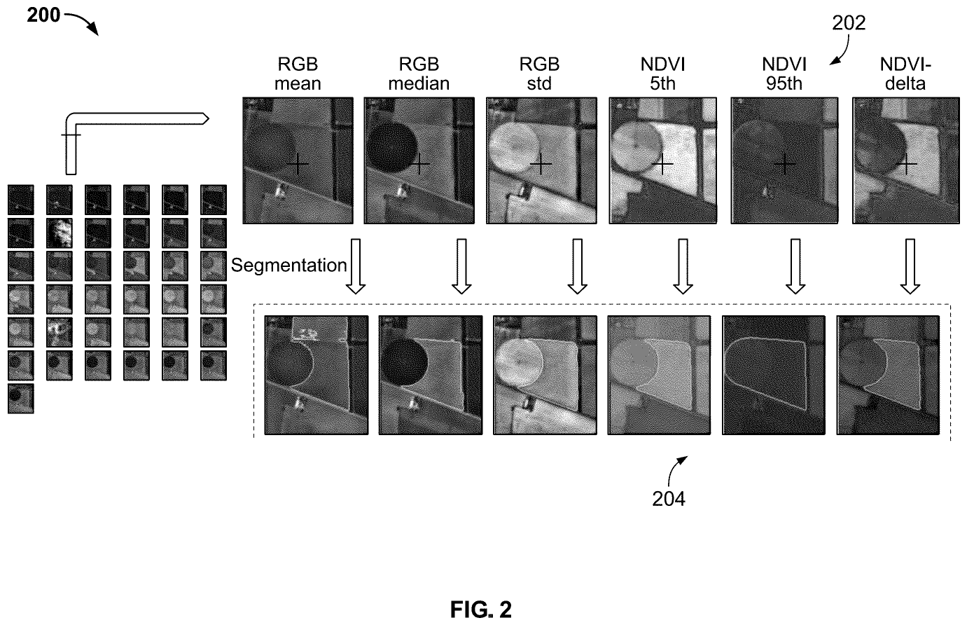

Next, in this example embodiment, the agricultural computer system 106 is configured, by the model 110, to output one or more segmentations, one for each of the composite images. The segmentations include an indicator of the field boundaries therein. FIG. 2, for example, illustrates the sequence 200 of the image data to the composites, as described above, and then to the output from the model 110. Specifically, the composite images include the composites for RGB mean, RGB median, RGB standard deviation, NVDI 5^{th}, NDVI 95^{th}, and NDVI-delta, as shown at 202. The segmentations generated by the model 110, then, are shown at 204. As shown, there is a segmentation generated, by the model 110, for each composite input to the model 110. As also apparent, the segmentation may vary among the different composites.

The agricultural computer system 106 is configured to then define the field boundary (ies) from the AOI based on the segmentation from the model 110. In doing so, the agricultural computer system 106 is configured, in this embodiment, to perform analysis of the representative segmentation (e.g., through overlaying the segmentations, etc.).

It should be understood, generally, that union of segmentations from the model 110 is indicative of L1, which is the boundary that detects/delineates overall administrative and/or management area of a field, which is stable across seasons, while the intersects of the segmentations is indicative of L2, which is the boundary that detects/delineates the actual cultivated area within the administrative and/or management area of a field, where non-planted area is excluded. In contrast to L1, L2 is seasonal specific. In this example embodiment, however, since discontinuity among pixels may be included in the different composites, the agricultural computer system 106 is configured to exclude over-segmentation and under-segmentation instances. Certain rules may be associated with the exclusion. For example, instances of over-segmentation may be identified based on segmentation contacting more than two sides of the AOI. In another example, instances of under-segmentation may be identified based on a segmentation area divided by the entire AOI area being less than a predefined threshold (e.g., 10% threshold, etc.).

It should be appreciated that the performance analysis may be based on other rules or criteria, for example, the AOI of the images, relative to the agricultural fields 102, etc.

In addition to the above, the agricultural computer system 106 is configured to assess the field boundaries defined by the composite segmentations and combinations thereof. In particular, ambiguity of the boundary (ies) may be indicated by the “stability of the boundaries”. The variable JC is employed to quantify the consistency of boundary demarcations across the composites. That is, where boundaries exhibit frequent alterations across different composites, such alterations may indicate a higher level of ambiguity and/or uncertainty, consequently implying lower reliability in the predictions (e.g., where JC<<1). Conversely, more consistent boundaries across composites may be indicative of stronger boundary information, leading to increased confidence in the predictive model (e.g., JC≈1).

In connection with the above, the overlay mask, or JC, herein, is defined as a sum of the segmentations from the composites, which is expressed in the equation below:

$$JC = \frac{1}{n} {\sum }_{i = 0} IoU ( mask = i , mask = i + 1 )$$

where n is the number of composites and IoU is the intersection over union of the mask i and the mask i+1, where i={0, 1, 2 . . . n}.

Consistent with the above, when the JC value exceeds a threshold, which indicates a stability of the boundary, the agricultural computer system 106 is configured to define the field boundary for the AOI as the intersection of the valid segmentation from the composites. The field boundary delineation is a set of polygons that defines the agricultural field(s) 102 in the AOI. The polygons may then be used to define the specific agricultural fields 102 to leverage any suitable digital agricultural product that requires accurate boundary information. This data can also be used to support precision harvest activities as the computing device 102 may be configured to export the polygons in multiple formats and store the same in geographic database systems.

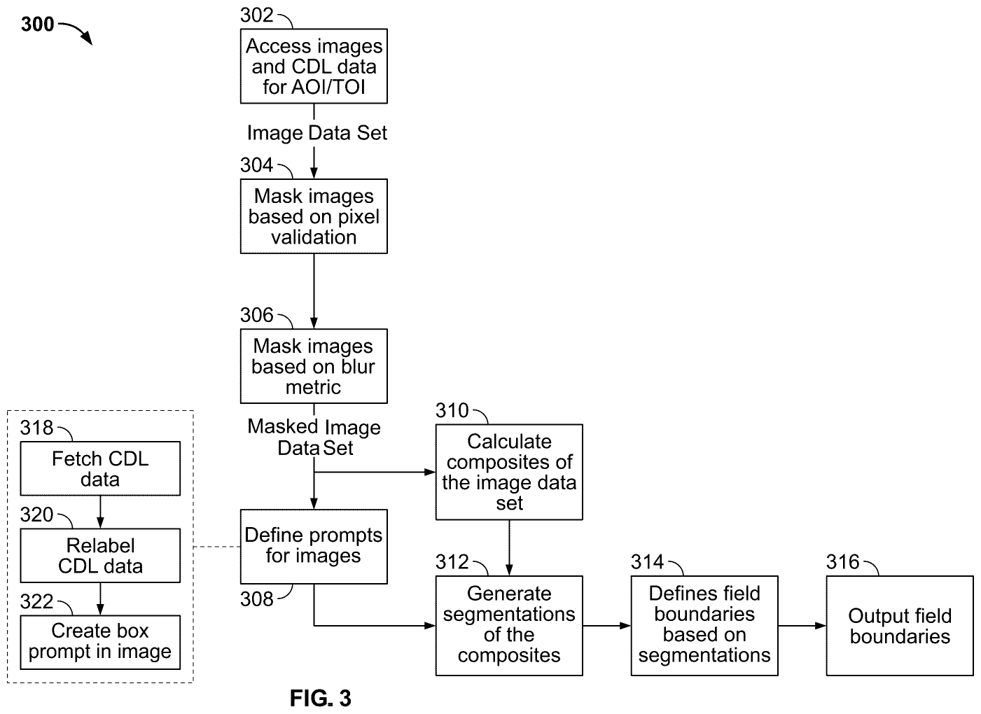

FIG. 3 illustrates an example method 300 for identifying field boundaries for agricultural fields based on images of the agricultural fields. The example method 300 is described herein in connection with the system 100, and may be implemented, in whole or in part, in the agricultural computer system 106 of the system 100. However, it should be appreciated that the method 300, or other methods described herein, are not limited to the system 100 or the agricultural computer system 106. And, conversely, the systems, data structures, and the computing devices described herein are not limited to the example method 300.

At the outset in the method 300, the agricultural computer system 106 is configured to identify an AOI/TOI and access, at 302, the image data from the data server 108 and the CDL data (e.g., from the USDA, etc.) consistent with the TOI and the AOI. The accessed image data may be referred to herein as an image data set. The image data set generally includes multiple pictures/images of the AOI, including at least one specific agricultural field (e.g., one of fields 102, etc.), and the TOI. For example the image data set may include all images of the agricultural field (from the data server 108, as captured by the satellite 104) between spring and fall of the given year, or multiple years.

At 304, the agricultural computer system 106 masks the images included in the image data set based on pixel validation. In particular, in this example, each pixel of each image is assigned a value, which is indicative of a content of the agricultural field represented by the pixel. For example, the pixel may be assigned: 2 for dark/shadow, 4 for vegetation, 5 for not-vegetation, 6 for water, and 7 for unclassified. The agricultural computer system 106 then counts the valid pixels, based on the assigned value, and compares the count to a threshold (e.g., 75%, 85%, 95%, etc.). The agricultural computer system 106 selects only the images having valid pixel counts that satisfy the threshold (e.g., above 85%, etc.), while other images are masked or filtered from the image data set.

At 306, the agricultural computer system 106 further masks the image data set based on “blurriness” of images. In particular, agricultural computer system 106 calculates a blur effective metric for each of the images included in the image data set. The agricultural computer system 106 selects only the images having a blur effect metric that satisfies a defined threshold (e.g., 0.3, etc.), while other images are masked or filtered from the image data set.

With continued reference to FIG. 3, at 308, the agricultural computer system 106 defines one or more prompts in the images. In one example, the prompt(s) may be defined based on edge detection within the image, where a centroid is placed and then either designated as the prompt or moved, based on identified edges, to become the prompt (e.g., a point prompt, etc.).

In some examples, the agricultural computer system 106 may define the one or more prompts in the images (at 308) by multiple sub-steps. That is, the agricultural computer system 106 identifies (or fetches), at 318, the CDL data (e.g., a map of such data, etc.) in the same resolution/project as the RGB data for the given image, or a different resolution than the image data (and modifies the same to approximate the resolution of the image data). In doing so, the agricultural computer system 106 may also ensure that the CDL data (e.g., the map, etc.) is pixel-level aligned with the given image, whether 1-to-1, or 1-to-N, where N is more than one, where the resolutions are different. In addition, the agricultural computer system 106 may also relabel, at 320, the CDL data for the field, for instance, to account for patches/areas in the field with the same crop type being different from each other (e.g., if they are separate patches, etc.). Then, for each crop patch, the agricultural computer system 106 creates, at 322, a box-prompt (having a box shape or other shape) within the image to represent the spatial extent of the crop patch.

It should be appreciated that any suitable technique may be employed to define prompt(s) within the background of the images, to avoid edges between ones of the agricultural fields, etc.

At 310, the agricultural computer system 106 calculates one or more different composites of the images included in the masked image data set. The composites may include, for example, RGB mean, RGB median, RGB standard deviation for the image pixels, the Normalized Difference Vegetation Index (NDVI), the Enhanced Vegetation Index 2 (EVI2), and the Modified Soil Adjusted Vegetation Index (MSAVI), etc., or derivations thereof (e.g., NVDI in the 5^{th} percentile, NDVI in the 95% percentile, delta between the 5^{th} percentile and in the 95^{th} percentile, etc.).

In this example embodiment, as explained above, the agricultural computer system 106 calculates six composite images from the mask image data set. At 312, the agricultural computer system 106 generates segmentations for each of the composites and the defined prompts. The segmentations are generated by the model 110, for example, again, with the composites and prompts as the inputs to the model. It should be appreciated, as above, that other models may be employed in generating segmentations in other method embodiments.

The agricultural computer system 106 then defines, at 314, the field boundary (ies) for the AOI based on the segmentations from the model 110 consistent with the description above. At 316, the agricultural computer system 106 outputs the filed boundary (ies) to the grower and/or one or more processes, which relies on the field boundaries.

With reference again to FIG. 1, as described, the data server 108 is communicatively coupled to the agricultural computer system 106 and is configured to send external data (e.g., data associated with growing spaces such as fields 102, or data as otherwise described herein, etc.) to agricultural computer system 106, via the data server 108 (or other data server) and the network(s) herein (e.g., for use with the model 110, etc.). The data server 108 may be owned or operated by the same legal person or entity as the agricultural computer system 106, or by a different person or entity, such as a government agency, non-governmental organization (NGO), and/or a private data service provider. Examples of external data may include location data, weather data, imagery data, soil data, seed data and treatment data as described herein, data from the various growing spaces herein, or statistical data relating to crop yields, among others (or other data as described herein). In some embodiments, the external data may also be provided by a data server owned by the same entity that owns and/or operates the agricultural computer system 106. For example, the agricultural computer system 106 may include a data server focused exclusively on a type of data that might otherwise be obtained from third party sources, such as weather data to trial data to treatment data. In some embodiments, data server 108 may be incorporated or integrated, in whole or in part, in the agricultural computer system 106.

Also, the fields 102 may have one or more remote sensors disposed thereon, where the sensor(s) are communicatively coupled, either directly or indirectly, to the agricultural computer system 106 and are configured to send sensor data to agricultural computer system 106.

In some examples, farm equipment may be included in the system 100 such as tractors, combines, pickers, sprayers, planters, trucks, fertilizer equipment, aerial vehicles including unmanned aerial vehicles, and any other item of physical machinery or hardware, typically mobile machinery, and which may be used in tasks associated with agriculture and/or related to operations described herein. In some embodiments, a single unit of the farm equipment may comprise a plurality of sensors that are coupled locally in a network on the apparatus/equipment. A controller area network (CAN) is an example of such a network that can be installed in combines, harvesters, sprayers, and cultivators. In connection therewith, then, an application controller associated with the farm equipment may be communicatively coupled to agricultural computer system via the network(s) and programmed, or configured, to receive one or more scripts (e.g., from the agricultural computer system, etc.) that are used to control an operating parameter of the farm equipment 106 (or another agricultural vehicle or implement). For instance, a CAN bus interface may be used to enable communications from the agricultural computer system 106 to the farm equipment 106 (e.g., to a computing device of the farm equipment, etc.), for example, such as through the CLIMATE FIELDVIEW DRIVE, available from Climate LLC, Saint Louis, Missouri. Sensor data may include the same type of information as field data. In some embodiments, remote sensors may not be fixed to the farm equipment 106 but may be remotely located in the field and may communicate with one or more networks of the system 100.

As indicated above, the network(s) of the system 100 are generally illustrated in FIG. 1 by arrowed lines. In connection therewith, the network(s) broadly represent any combination of one or more data communication networks including local area networks, wide area networks, internetworks or internets, using any of wireline or wireless links, including terrestrial or satellite links. The network(s) may be implemented by any medium or mechanism that provides for the exchange of data between the various elements of FIG. 1. The various elements of FIG. 1 may also have direct (wired or wireless) communications links. For instance, the data server 108, the agricultural computer system 106, and other elements of the system 100 may each comprise an interface compatible with the network(s) and programmed, or configured, to use standardized protocols for communication across the networks, such as TCP/IP, Bluetooth, CAN protocol and higher-layer protocols, such as HTTP, TLS, and the like.

That said, the agricultural computer system 106 is configured, generally, to receive data from farm the fields 102, the satellite 104, and/or other sources. Agricultural computer system 106 may be further configured to host, use or execute one or more computer programs, other software elements, digitally programmed logic, such as FPGAs or ASICs, or any combination thereof, to perform translation and storage of data values, construction of digital models of one or more crops on one or more fields, generation of recommendations and notifications, and generation and sending of scripts, in the manner described further in other sections of this disclosure.

In an embodiment, agricultural computer system 106 is programmed with or comprises a communication layer 132, a presentation layer 134, a data management layer 140, a hardware/virtualization layer 150, and a data repository layer 160. “Layer,” in this context, refers to any combination of electronic digital interface circuits, microcontrollers, firmware, such as drivers, and/or computer programs, or other software elements.

Communication layer 132 may be configured to perform input/output interfacing functions including sending requests to the data server 108 and/or to remote sensor(s) for field data from the field, etc. Communication layer 132 may be configured to send the received data to the data repository layer 160 to be stored (e.g., in agricultural computer system 106, etc.). Presentation layer 134 may be configured to generate a graphical user interface (GUI) to be displayed on a communication device, via one or more applications (e.g., to interact with the agricultural computer system 106, etc.), or other computers that are coupled to the agricultural computer system 106 through the network(s). The GUI may comprise controls for inputting data to be sent to the agricultural computer system 106, generating requests for models and/or recommendations, and/or displaying recommendations, notifications, models, and other data.

Data management layer 140 may be configured to manage read operations and write operations involving the repository layer 160 and other functional elements of the system 100, including queries and result sets communicated between the functional elements of the system and the repository layer 160. Examples of data management layer 140 include JDBC, SQL server interface code, and/or HADOOP interface code, among others. The repository layer 160 may comprise a database. As used herein, the term “database” may refer to either a body of data, a relational database management system (RDBMS), or both. As used herein, a database may comprise any collection of data including hierarchical databases, relational databases, flat file databases, object-relational databases, object oriented databases, distributed databases, and any other structured collection of records or data that is stored in a computer system. Examples of RDBMS's include, but are not limited to, ORACLE®, MYSQL, IBM® DB2, MICROSOFT® SQL SERVER, SYBASE®, and POSTGRESQL databases. That said, any database may be used that enables the systems and methods described herein.

When data is not provided directly to the agricultural computer system 106, for example, via sensors, the satellite 104, etc., a user may be prompted via one or more user interfaces on a communication device (served by the agricultural computer system 106) to input such data to the agricultural computer system 106. In an example embodiment, the user may specify an AOI by accessing a map on the communication device (served by the agricultural computer system 106) and selecting desired locations, for example, of the fields 102.

In an example embodiment, the agricultural computer system 106 is programmed to generate and cause displaying of one or more interfaces comprising a data manager for data input. After one or more fields (or associated data) have been identified using the methods described above, the data manager may provide one or more graphical user interface widgets, which when selected can identify field boundaries identified by the disclosure herein for fields of the growing spaces, etc. The data manager may include a text view, a graphical view, and/or one or more editable programs.

In an embodiment, the above described model 110 and data is stored in the repository layer 160. “Model,” in this context, refers to an electronic digitally stored set of executable instructions and data values, associated with one another, which are capable of receiving and responding to a programmatic or other digital call, invocation, or request for resolution based upon specified input values, to yield one or more stored or calculated output values indicative of field boundaries that can serve as the basis of computer-implemented output data displays, or machine control, among other things.

With continued reference to FIG. 1, in an embodiment, instructions 135 of the agricultural computer system 106 may comprise a set of one or more pages of main memory, such as RAM, in the agricultural computer system 106 into which executable instructions have been loaded and which when executed cause the agricultural computer system 106 to perform the functions or operations that are described herein. For example, the instructions 135 may comprise a set of pages in RAM that contain instructions which, when executed, cause determining likelihoods of occurrence of one or more diseases as described herein. The instructions may be in machine executable code in the instruction set of a CPU and may have been compiled based upon source code written in JAVA, C, C++, OBJECTIVE-C, or any other human-readable programming language or environment, alone or in combination with scripts in JAVASCRIPT, other scripting languages and other programming source text. The term “pages” is intended to refer broadly to any region within main memory and the specific terminology used in a system may vary depending on the memory architecture or processor architecture. In another embodiment, the instructions 135 also may represent one or more files, or projects of source code, that are digitally stored in a mass storage device, such as non-volatile RAM or disk storage, in the agricultural computer system 106 or a separate repository system, which when compiled or interpreted cause generating executable instructions which when executed cause the agricultural computer system 106 to perform the functions or operations that are described herein.

Hardware/virtualization layer 150 comprises one or more central processing units (CPUs), memory controllers, and other devices, components, or elements of a computer system, such as volatile or non-volatile memory, non-volatile storage, such as disk, and I/O devices or interfaces, etc. The hardware/virtualization layer 150 also may comprise programmed instructions that are configured to support visualization, virtualization, containerization, or other technologies.

For purposes of illustrating a clear example, FIG. 1 shows a limited number of instances of certain functional elements. However, in other embodiments, there may be any number of such elements. For example, embodiments may use thousands or millions of different mobile computing devices associated with different users/growers. Further, the agricultural computer system 106 and/or data server 108 may be implemented using two or more processors, cores, clusters, or instances of physical machines or virtual machines, configured in a discrete location or co-located with other elements in a datacenter, shared computing facility or cloud computing facility.

In an embodiment, the implementation of the functions described herein using one or more computer programs, or other software elements that are loaded into and executed using one or more general-purpose computers, will cause the general-purpose computers to be configured as a particular machine or as a computer that is specially adapted to perform the functions described herein. Further, each of the flow diagrams that are described further herein may serve, alone or in combination with the descriptions of processes and functions in prose herein, as algorithms, plans or directions that may be used to program a computer or logic to implement the functions that are described. In other words, all the prose text herein, and all the drawing figures, together are intended to provide disclosure of algorithms, plans or directions that are sufficient to permit a skilled person to program a computer to perform the functions that are described herein, in combination with the skill and knowledge of such a person given the level of skill that is appropriate for disclosures of this type.

In an embodiment, a user interacts with the agricultural computer system 106 using a communication device (or other computing device) configured with an operating system and one or more applications or apps. The communication device also may interoperate with the agricultural computer system 106 independently and automatically under program control or logical control and direct user interaction is not always required. The communication device broadly represents one or more of a smart phone, PDA, tablet computing device, laptop computer, desktop computer, workstation, or any other computing device capable of transmitting and receiving information and performing the functions described herein. The communication device may communicate via a network using a mobile application stored on the communication device, and in some embodiments, the communication device may be coupled using a cable or connector to one or more sensors and/or other apparatus in the system 100. The particular user may own, operate or possess and use, in connection with system 100, more than one communication device at a time.

The application associated with the communication device may provide client-side functionality, via the network to one or more mobile computing devices. Again, the communication device may access the application, via a web browser or a local client application or app. The communication device may transmit data to, and receive data from, one or more front-end servers, using web-based protocols, or formats, such as HTTP, XML and/or JSON, or app-specific protocols. In an example embodiment, the data may take the form of requests (e.g., filter criteria, selections, etc.) and user information input, such as data (e.g., disease observation, etc.), into the communication device 104.

A commercial example of the application described above is CLIMATE FIELDVIEW, commercially available from Climate LLC, Saint Louis, Missouri. The CLIMATE FIELDVIEW application and associated tools, or other applications, may be modified, extended, or adapted to include features, functions, and programming that have not been disclosed earlier than the filing date of this disclosure. In one embodiment, the application comprises an integrated software platform that allows a grower to make fact-based decisions for their operation because it combines historical data about the grower's fields with any other data that the grower wishes to compare. The combinations and comparisons may be performed in real time and are based upon scientific models that provide potential scenarios to permit the grower to make better, more informed decisions.

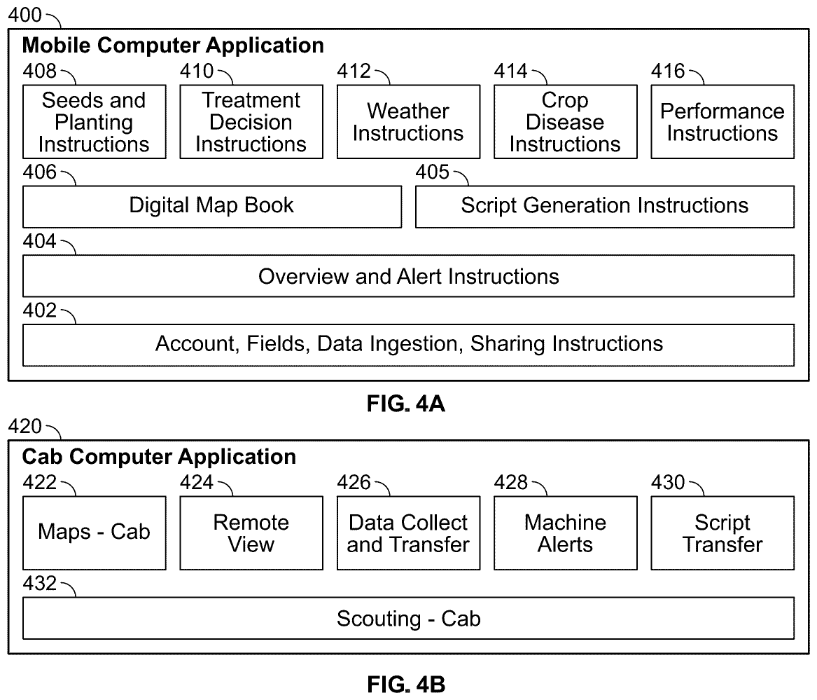

FIGS. 4A-4B illustrate two views of an example logical organization of sets of instructions in main memory when an example mobile application is loaded for execution. Each named element represents a region of one or more pages of RAM, or other main memory, or one or more blocks of disk storage, or other non-volatile storage, and the programmed instructions within those regions. In one embodiment, in FIG. 4A, a mobile computer application 400 comprises account, fields, data ingestion, sharing instructions 402, overview and alert instructions 404, digital map book instructions 406, seeds and planting instructions 408, treatment decision instructions 410, weather instructions 412, crop disease type instructions 414, and performance instructions 416.

In one embodiment, a mobile computer application 400 comprises account, fields, data ingestion, sharing instructions 402 which are programmed to receive, translate, and ingest field data from third party systems via manual upload or APIs. Data types may include field boundaries, yield maps, as-planted maps, soil test results, as-applied maps, and/or management zones, among others. Data formats may include shape files, native data formats of third parties, and/or farm management information system (FMIS) exports, among others. Receiving data may occur via manual upload, e-mail with attachment, external APIs that push data to the mobile application, or instructions that call APIs of external systems to pull data into the mobile application. In one embodiment, mobile computer application 400 comprises a data inbox. In response to receiving a selection of the data inbox, the mobile computer application 400 may display a graphical user interface for manually uploading data files and importing uploaded files to a data manager.

In one embodiment, digital map book instructions 406 comprise field map data layers stored in device memory and are programmed with data visualization tools and geospatial field notes. This provides growers with convenient information close at hand for reference, logging and visual insights into field performance and other options provided herein. In one embodiment, overview and alert instructions 404 are programmed to provide an operation-wide view of what is important to the grower, and timely recommendations to take action or focus on particular issues. This permits the grower to focus time on what needs attention, to save time and preserve yield throughout the season. In one embodiment, seeds and planting instructions 408 are programmed to provide tools for seed selection, hybrid placement, and script creation, including variable rate (VR) script creation, based upon scientific models and empirical data. This enables growers to maximize yield, or return on investment, through optimized seed purchase, placement and population.

In one embodiment, script generation instructions 405 are programmed to provide an interface for generating scripts, including variable rate (VR) fertility scripts, disease treatment scripts, etc. The interface enables growers to create scripts for field implements, such as treatment decisions, planting, and irrigation. For example, a planting script interface may comprise tools for identifying a type of seed for planting. Upon receiving a selection of the seed type, mobile computer application 400 may display one or more fields broken into management zones, such as the field map data layers created as part of digital map book instructions 406. In one embodiment, the management zones comprise soil zones along with a panel identifying each soil zone and a soil name, texture, drainage for each zone, or other field data. Mobile computer application 400 may also display tools for editing or creating such as graphical tools for drawing management zones, such as soil zones, over a map of one or more fields. Planting procedures may be applied to all management zones or different planting procedures may be applied to different subsets of management zones. When a script is created, mobile computer application 400 may make the script available for download in a format readable by an application controller, such as an archived or compressed format. Additionally, and/or alternatively, a script may be sent directly to a cab computer (of farm equipment) from mobile computer application 400 and/or uploaded to one or more data servers and stored for further use.

In one embodiment, treatment decision instructions 410 are programmed to provide tools to inform decisions by visualizing or instruction about the application of one or more candidate treatments to crops in a particular field. This enables growers to potentially enhance yield or return on investment through treatment application during the season.

Example programmed functions include displaying images to enable tuning application(s) of treatment across multiple zones; output of scripts to drive machinery; tools for mass data entry and adjustment; and/or maps for data visualization, among others. Treatment decision instructions 410 also may be programmed to generate and cause displaying a treatment graph, indicative of the application of the treatment to one or more target fields, but not others based on the functions explained herein. In one embodiment, the treatment graph may include one or more user input features, such as dials or slider bars, to dynamically change the candidate treatment programs so that the grower may alter the parameters of the treatment decision. Treatment instructions 410 also may be programmed to generate and cause displaying a treatment decision or indications.

In one embodiment, weather instructions 412 are programmed to provide field-specific recent weather data and forecasted weather information. This enables growers to save time and have an efficient integrated display with respect to daily operational decisions.

In one embodiment, crop disease type instructions 414 are programmed to provide timely remote sensing images highlighting in-season crop variation, multiple crop disease types and potential concerns. Example programmed functions include cloud checking, to identify possible clouds or cloud shadows; determining treatment indices based on field images; graphical visualization of scouting layers, including, for example, those related to field health, and viewing and/or sharing of scouting notes; recording observations of different crop disease type presence and/or severity; and/or downloading satellite images from multiple sources and prioritizing the images for the grower, among others.

In one embodiment, performance instructions 416 are programmed to provide reports, analysis, and insight tools using on-farm data for evaluation, insights and decisions. This enables the grower to seek improved outcomes for the next year through fact-based conclusions about why return on investment was at prior levels, and insight into yield-limiting factors. The performance instructions 416 may be programmed to communicate via the network(s) to back-end analytics programs executed at agricultural computer system 106 and/or data server 108 and configured to analyze metrics, such as yield, yield differential, hybrid, population, SSURGO zone, soil test properties, or elevation, among others. Programmed reports and analysis may include yield variability analysis, treatment effect estimation, benchmarking of yield and other metrics against other growers based on anonymized data collected from many growers, or data for seeds and planting, among others.

Applications having instructions configured in this way may be implemented for different computing device platforms while retaining the same general user interface appearance. For example, the mobile application may be programmed for execution on tablets, smartphones, or server computers that are accessed using browsers at client computers. Further, the mobile application 400 as configured for tablet computers, or smartphones, may provide a full app experience, or a cab app experience, that is suitable for the display and processing capabilities of a cab computer. For example, referring now to FIG. 4B, in one embodiment a cab computer application 420 (e.g., as accessible in one of farm equipment 106, etc.) may comprise maps-cab instructions 422, remote view instructions 324, data collect and transfer instructions 426, machine alerts instructions 428, script transfer instructions 430, and scouting-cab instructions 432. The code base for the instructions of FIG. 4B may be the same as for FIG. 4A and executables implementing the code may be programmed to detect the type of platform on which they are executing and to expose, through a graphical user interface, only those functions that are appropriate to a cab platform or full platform. This approach enables the system to recognize the distinctly different user experience that is appropriate for an in-cab environment and the different technology environment of the cab. The maps-cab instructions 422 may be programmed to provide map views of fields, farms or regions that are useful in directing machine operation. The remote view instructions 424 may be programmed to turn on, manage, and provide views of machine activity in real-time or near real-time to other computing devices connected to the agricultural computer system 106 via wireless networks, wired connectors or adapters, and the like. The data collect and transfer instructions 426 may be programmed to turn on, manage, and provide transfer of data collected at sensors and controllers to the agricultural computer system 106 via wireless networks, wired connectors or adapters, and the like (e.g., via network(s) in the system 100, etc.). The machine alerts instructions 428 may be programmed to detect issues with operations of the machines or tools that are associated with the cab and generate operator alerts. The script transfer instructions 430 may be configured to transfer in scripts of instructions that are configured to direct machine operations or the collection of data. The scouting-cab instructions 432 may be programmed to display location-based alerts and information received from the agricultural computer system 106 based on the location of a communication device, farm equipment, or sensors in the field (of the growing spaces) and ingest, manage, and provide transfer of location-based scouting observations to the agricultural computer system 106 based on the location of the farm equipment, or sensors in the field.

In an embodiment, data server 108 stores data including soil data representing soil composition for the one or more fields and weather data representing temperature and precipitation on the one or more fields (and/or other data). The weather data may include past and present weather data as well as forecasts for future weather data. In an embodiment, data server 108 comprises a plurality of servers hosted by different entities. For example, a first server may contain soil composition data while a second server may include weather data. Additionally, soil composition data may be stored in multiple servers. For example, one server may store data representing percentage of sand, silt, and clay in the soil while a second server may store data representing percentage of organic matter (OM) in the soil. Further, in some embodiments, the data server 108, again, may include data associated with the growing spaces with regard to available seeds for use in comparisons, etc.

In an embodiment, remote sensors in the system 100 may comprise one or more sensors that are programmed, or configured, to produce one or more observations related to growing spaces, trials therein, etc. Remote sensors may be aerial sensors, such as satellites, vehicle sensors, planting equipment sensors, tillage sensors, fertilizer or insecticide application sensors, harvester sensors, and any other implement capable of receiving data from the one or more fields (e.g., associated with one or more of the growing spaces, etc.). In an embodiment, farm equipment may include an application controller programmed, or configured, to receive instructions from agricultural computer system 106. The application controller may also be programmed, or configured, to control an operating parameter of the farm equipment. Other embodiments may use any combination of sensors and controllers, of which the following are merely selected examples.

The system 100 may obtain or ingest data under grower control, on a mass basis from a large number of growers who have contributed trial data or other data to a shared database system. This form of obtaining data may be termed “manual data ingest” as one or more user-controlled computer operations are requested, or triggered, to obtain data for use by the agricultural computer system 106. As an example, the CLIMATE FIELDVIEW application, commercially available from Climate LLC, Saint Louis, Missouri, may be operated to export data to agricultural computer system 106 for storing in the field data repository 160.

For example, seed monitor systems can both control planter apparatus components and obtain planting data, including signals from seed sensors via a signal harness that comprises a CAN backbone and point-to-point connections for registration and/or diagnostics. Seed monitor systems can be programmed, or configured, to display seed spacing, population and other information to the user via a cab computer of the apparatus, or other devices within the system 100.

Likewise, yield monitor systems may contain yield sensors for harvester apparatus that send yield measurement data to a cab computer of the apparatus, or other devices within the system 100. Yield monitor systems may utilize one or more remote sensors to obtain grain moisture measurements in a combine, or other harvester, and transmit these measurements to the user 103 via the cab computer, or other devices within the system 100.

In an embodiment, examples of sensors that may be used with any moving vehicle, or apparatus of the type described elsewhere herein, include kinematic sensors and position sensors. Kinematic sensors may comprise any of speed sensors, such as radar or wheel speed sensors, accelerometers, or gyros. Position sensors may comprise GPS receivers or transceivers, or WiFi-based position or mapping apps that are programmed to determine location based upon nearby WiFi hotspots, among others.

In an embodiment, examples of sensors that may be used with tractors, or other moving vehicles, include engine speed sensors, fuel consumption sensors, area counters or distance counters that interact with GPS or radar signals, PTO (power take-off) speed sensors, tractor hydraulics sensors configured to detect hydraulics parameters, such as pressure or flow, and/or and hydraulic pump speed, wheel speed sensors or wheel slippage sensors. In an embodiment, examples of controllers that may be used with tractors include hydraulic directional controllers, pressure controllers, and/or flow controllers; hydraulic pump speed controllers; speed controllers or governors; hitch position controllers; or wheel position controllers provide automatic steering.

In an embodiment, examples of sensors that may be used with seed planting equipment, such as planters, drills, or air seeders, include seed sensors, which may be optical, electromagnetic, or impact sensors; downforce sensors, such as load pins, load cells, pressure sensors; soil property sensors, such as reflectivity sensors, moisture sensors, electrical conductivity sensors, optical residue sensors, or temperature sensors; component operating criteria sensors, such as planting depth sensors, downforce cylinder pressure sensors, seed disc speed sensors, seed drive motor encoders, seed conveyor system speed sensors, or vacuum level sensors; or pesticide application sensors, such as optical or other electromagnetic sensors, or impact sensors. In an embodiment, examples of controllers that may be used with such seed planting equipment include: toolbar fold controllers, such as controllers for valves associated with hydraulic cylinders; downforce controllers, such as controllers for valves associated with pneumatic cylinders, airbags, or hydraulic cylinders, and programmed for applying downforce to individual row units or an entire planter frame; planting depth controllers, such as linear actuators; metering controllers, such as electric seed meter drive motors, hydraulic seed meter drive motors, or swath control clutches; hybrid selection controllers, such as seed meter drive motors, or other actuators programmed for selectively allowing or preventing seed or an air-seed mixture from delivering seed to or from seed meters or central bulk hoppers; metering controllers, such as electric seed meter drive motors, or hydraulic seed meter drive motors; seed conveyor system controllers, such as controllers for a belt seed delivery conveyor motor; marker controllers, such as a controller for a pneumatic or hydraulic actuator; or pesticide application rate controllers, such as metering drive controllers, orifice size or position controllers.

In an embodiment, examples of sensors that may be used with tillage equipment include position sensors for tools, such as shanks or discs; tool position sensors for such tools that are configured to detect depth, gang angle, or lateral spacing; downforce sensors; or draft force sensors. In an embodiment, examples of controllers that may be used with tillage equipment include downforce controllers or tool position controllers, such as controllers configured to control tool depth, gang angle, or lateral spacing.

In an embodiment, examples of sensors that may be used in relation to an apparatus for applying fertilizer, insecticide, fungicide, herbicide, and the like, such as on-planter starter fertilizer systems, subsoil fertilizer applicators, or fertilizer sprayers, include: fluid system criteria sensors, such as flow sensors or pressure sensors; sensors indicating which spray head valves or fluid line valves are open; sensors associated with tanks, such as fill level sensors; sectional or system-wide supply line sensors, or row-specific supply line sensors; or kinematic sensors, such as accelerometers disposed on sprayer booms. In an embodiment, examples of controllers that may be used with such apparatus include pump speed controllers; valve controllers that are programmed to control pressure, flow, direction, PWM and the like; or position actuators, such as for boom height, subsoiler depth, or boom position.

In an embodiment, examples of sensors that may be used with harvesters include yield monitors, such as impact plate strain gauges or position sensors, capacitive flow sensors, load sensors, weight sensors, or torque sensors associated with elevators or augers, or optical or other electromagnetic grain height sensors; grain moisture sensors, such as capacitive sensors; grain loss sensors, including impact, optical, or capacitive sensors; header operating criteria sensors, such as header height, header type, deck plate gap, feeder speed, and reel speed sensors; separator operating criteria sensors, such as concave clearance, rotor speed, shoe clearance, or chaffer clearance sensors; auger sensors for position, operation, or speed; or engine speed sensors. In an embodiment, examples of controllers that may be used with harvesters include header operating criteria controllers for elements, such as header height, header type, deck plate gap, feeder speed, or reel speed; separator operating criteria controllers for features such as concave clearance, rotor speed, shoe clearance, or chaffer clearance; or controllers for auger position, operation, or speed.

In an embodiment, examples of sensors that may be used with grain carts include weight sensors, or sensors for auger position, operation, or speed. In an embodiment, examples of controllers that may be used with grain carts include controllers for auger position, operation, or speed.

In an embodiment, examples of sensors and controllers may be installed in unmanned aerial vehicle (UAV) apparatus or “drones.” Such sensors may include cameras with detectors effective for any range of the electromagnetic spectrum including visible light, infrared, ultraviolet, near-infrared (NIR), and the like; accelerometers; altimeters; temperature sensors; humidity sensors; pitot tube sensors or other airspeed or wind velocity sensors; battery life sensors; or radar emitters and reflected radar energy detection apparatus; other electromagnetic radiation emitters and reflected electromagnetic radiation detection apparatus. Such controllers may include guidance or motor control apparatus, control surface controllers, camera controllers, or controllers programmed to turn on, operate, obtain data from, manage and configure any of the foregoing sensors.

In an embodiment, sensors and controllers may be affixed to a soil sampling and measurement apparatus that is configured, or programmed, to sample soil and perform soil chemistry tests, soil moisture tests, and other tests pertaining to soil.

In an embodiment, sensors and controllers may comprise weather devices for monitoring weather conditions of fields.

In an embodiment, the agricultural computer system 106 is programmed, or configured, to create an agronomic model. In this context, an agronomic model is a data structure in memory of the agricultural computer system 106 that comprises field data, such as identification data and harvest data for one or more fields. The agronomic model may also comprise calculated agronomic properties which describes either conditions which may affect the growth of one or more crops on a field, or properties of the one or more crops, or both. Additionally, an agronomic model may comprise recommendations based on agronomic factors, such as crop recommendations, irrigation recommendations, planting recommendations, fertilizer recommendations, fungicide recommendations, pesticide recommendations, harvesting recommendations and other crop management recommendations. The agronomic factors may also be used to estimate one or more crop related results, such as agronomic yield. The agronomic yield of a crop is an estimate of quantity of the crop that is produced, or in some examples, the revenue or profit obtained from the produced crop.

In an embodiment, the agricultural computer system 106 may use a preconfigured agronomic model to calculate agronomic properties related to currently received location and crop information for one or more fields. The preconfigured agronomic model is based upon previously processed field data, including but not limited to, identification data, harvest data, fertilizer data, and weather data. The preconfigured agronomic model may have been cross validated to ensure accuracy of the model. Cross validation may include comparison to ground truthing that compares predicted results with actual results on a field, such as a comparison of precipitation estimate with a rain gauge or sensor providing weather data at the same or nearby location, or a comparison of treatment recommendations to validation data.

According to one example embodiment, the techniques described herein are implemented by one or more special-purpose computing devices. The special-purpose computing devices may be hard-wired to perform the techniques, or may include digital electronic devices, such as one or more application-specific integrated circuits (ASICs) or field programmable gate arrays (FPGAs), that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be a desktop computer systems, portable computer systems, handheld devices, networking devices or any other device that incorporates hard-wired and/or program logic to implement the techniques.

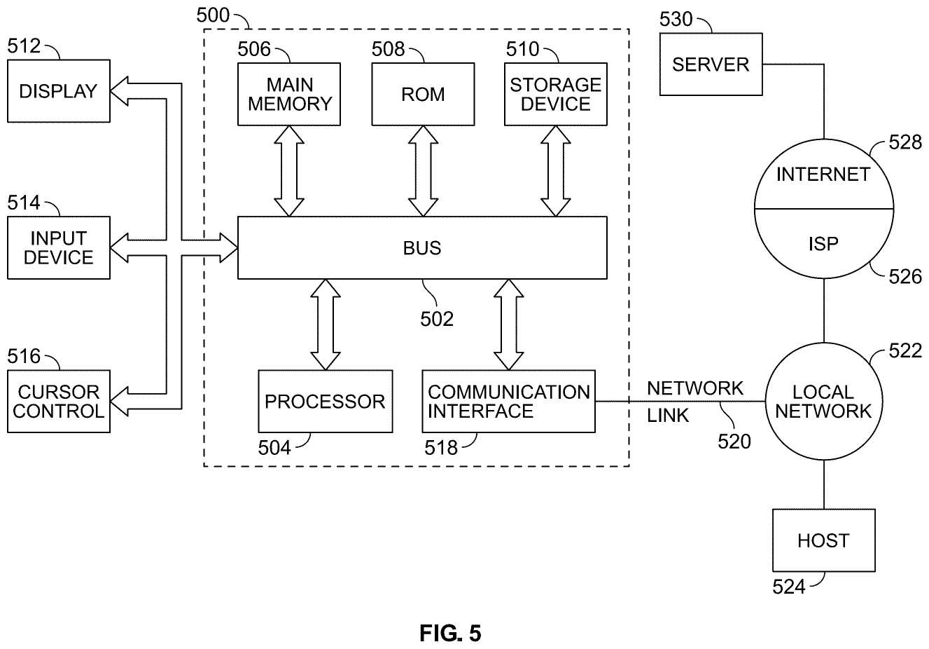

For example, FIG. 5 is a block diagram that illustrates a computer system 500 upon which embodiments of the present disclosure may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508, or other static storage device coupled to bus 502, for storing static information and instructions for processor 504. A storage device 510, such as a magnetic disk, optical disk, or solid-state drive, is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes; a first axis (e.g., x, etc.) and a second axis (e.g., y, etc.), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of, or in combination with, software instructions.

The term “storage media” as used herein refers to any non-transitory media that stores data and/or instructions that cause a machine to operate in a specific fashion. Such storage media may comprise non-volatile media and/or volatile media. Non-volatile media includes, for example, optical disks, magnetic disks, or solid-state drives, such as storage device 510. Volatile media includes dynamic memory, such as main memory 506. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from, but may be used in conjunction with, transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infrared data communications.

Various forms of media may be involved in carrying one or more sequences of one or more instructions to processor 504 for execution. For example, the instructions may initially be carried on a magnetic disk or solid-state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infrared signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

With that said, it should be appreciated that the functions described herein, in some embodiments, may be described in computer executable instructions stored on a computer readable media, and executable by one or more processors. The computer readable media is a non-transitory computer readable media. By way of example, and not limitation, such computer readable media can include RAM, ROM, EEPROM, CD-ROM or other optical disk storage, magnetic disk storage or other magnetic storage device, or any other medium that can be used to carry or store desired program code in the form of instructions or data structures and that can be accessed by a computer. Combinations of the above should also be included within the scope of computer-readable media.

It should also be appreciated that one or more aspects of the present disclosure transform a general-purpose computing device into a special-purpose computing device when configured to perform the functions, methods, and/or processes described herein.

As will be appreciated based on the foregoing specification, the above-described embodiments of the disclosure may be implemented using computer programming or engineering techniques including computer software, firmware, hardware or any combination or subset thereof, wherein the technical effect may be achieved by performing at least one of the following operations: a) accessing an image data set, which includes multiple images of an agricultural field; b) masking the image data set based on one or more criteria; c) calculating, by a computing device, one or more composites from the image data set; d) generating, by the computing device, using a model, a segmentation of each of the one or more composites, based on the one or more composites; and e) combining the segmentation(s) for the one or more composites into a field boundary for the agricultural field.

Examples and embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail. In addition, advantages and improvements that may be achieved with one or more example embodiments disclosed herein may provide all or none of the above mentioned advantages and improvements and still fall within the scope of the present disclosure.

Specific values disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may also be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on,” “engaged to,” “connected to,” “coupled to,” “associated with,” “in communication with,” or “included with” another element or layer, it may be directly on, engaged, connected or coupled to, or associated or in communication or included with the other feature, or intervening features may be present. As used herein, the term “and/or” and the phrase “at least one of” includes any and all combinations of one or more of the associated listed items.

Although the terms first, second, third, etc. may be used herein to describe various features, these features should not be limited by these terms. These terms may be only used to distinguish one feature from another. Terms such as “first,” “second,” and other numerical terms when used herein do not imply a sequence or order unless clearly indicated by the context. Thus, a first feature discussed herein could be termed a second feature without departing from the teachings of the example embodiments.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for processing images related to boundaries, the computer-implemented method comprising:
accessing an image data set, which includes multiple images of an agricultural field;
masking the image data set based on one or more criteria;
calculating, by a computing device, one or more composites from the image data set;
generating, by the computing device, using a model, a segmentation of each of the one or more composites, based on the one or more composites; and
combining the segmentation(s) for the one or more composites into a field boundary for the agricultural field.

2. The computer-implemented method of claim 1, wherein the multiple images include multiple images over a time of interest (TOI), which includes multiple seasons of a calendar year.

3. The computer-implemented method of claim 1, wherein the one or more criteria includes a pixel validation and a blur effective metric, relative to at least one threshold.

4. The computer-implemented method of claim 1, wherein the one or more composites include an RGB mean composite, a RGB median composite, and/or RGB standard deviation composite; and/or
wherein the one or more composites include an NVDI in the 5^{th} percentile composite, an NDVI in the 95% percentile composite, and/or a delta composite between the 5^{th} percentile and in the 95^{th} percentile.

5. The computer-implemented method of claim 1, wherein the model includes a segment anything model.

6. The computer-implemented method of claim 5, further comprising defining one or more prompts for the image data set; and
wherein generating the segments for each of the one or more composites is further based on the defined one or more prompts.

7. The computer-implemented method of claim 6, further comprising accessing crop-specific land cover data for the image data set; and
wherein the one or more prompts includes a bounding-box prompt.

8. The computer-implemented method of claim 1, wherein combining the segmentation(s) includes combining the segmentation(s) based on one or more rules related to under-segmentation, over-segmentation, and/or consistency of boundary demarcations across the one or more composites.

9. A system for processing images related to boundaries, the system comprising at least one computing device configured to:
access an image data set, which includes multiple images of an agricultural field;
mask the image data set based on one or more criteria;
calculate one or more composites from the image data set;
generate, using a model, a segmentation of each of the one or more composites, based on the one or more composites; and
combine the segmentation(s) for the one or more composites into a field boundary for the agricultural field.

10. The system of claim 9, wherein the multiple images include multiple images over a time of interest (TOI), which includes multiple seasons of a calendar year.

11. The system of claim 9, wherein the one or more criteria includes a pixel validation and a blur effective metric, relative to at least one threshold.

12. The system of claim 9, wherein the one or more composites include an RGB mean composite, a RGB median composite, and/or RGB standard deviation composite; and/or wherein the one or more composites include an NVDI in the 5^{th} percentile composite, an NDVI in the 95% percentile composite, and/or a delta composite between the 5^{th} percentile and in the 95^{th} percentile.

13. The system of claim 9, wherein the model includes a segment anything model.

14. The system of claim 13, wherein the at least one computing device is further configured to define one or more prompts for the image data set; and
wherein the at least one computing device is configured, in order to generate the segments for each of the one or more composites, to generate the segments further based on the defined one or more prompts.

15. The system of claim 14, wherein the at least one computing device is further configured to access crop-specific land cover data for the image data set; and
wherein the one or more prompts includes a bounding-box prompt.

16. The system of claim 9, wherein the at least one computing device is configured, in order to combine the segmentation(s), to combine the segmentation(s) based on one or more rules related to under-segmentation, over-segmentation, and/or consistency of boundary demarcations across the one or more composites.

17. One or more non-transitory computer-readable media including executable instructions for processing images related to boundaries, which when executed by at least one processor, cause the at least one processer to:
access an image data set, which includes multiple images of an agricultural field;
mask the image data set based on one or more criteria;
calculate one or more composites from the image data set;
generate, using a model, a segmentation of each of the one or more composites, based on the one or more composites; and
combine the segmentation(s) for the one or more composites into a field boundary for the agricultural field.

18. The one or more non-transitory computer-readable media of claim 17, wherein the multiple images include multiple images over a time of interest (TOI), which includes multiple seasons of a calendar year; and
wherein the one or more criteria includes a pixel validation and a blur effective metric, relative to at least one threshold.

19. The one or more non-transitory computer-readable media of claim 17, wherein the one or more composites include an RGB mean composite, a RGB median composite, and/or RGB standard deviation composite; and/or
wherein the one or more composites include an NVDI in the 5^{th} percentile composite, an NDVI in the 95% percentile composite, and/or a delta composite between the 5^{th} percentile and in the 95^{th} percentile.

20. The one or more non-transitory computer-readable media of claim 17, wherein the model includes a segment anything model; and
wherein the executable instructions, when executed by the at least one processor, further cause the at least one processer to:
access crop-specific land cover data for the image data set;
define one or more bounding-box prompts for the image data set; and
generate the segments further based on the defined one or more bounding-box prompts.
